(19) United States
(12) Patent Application Publication
(10) Pub. No.: US 2007/0287871 A1
(43) Pub. Date: Dec. 13, 2007

(54) Silicoaluminophosphate isomerization catalyst

(76) Inventors: Eelko Brevoord, Amersfoort (NL); Stephan Janbroers, Diemen (NL); Mark Hendrikus Harte, Zaandam (NL); Pekka Aalto, Porvoo (FI); Elina Harlin, Vantaa (FI); Heidi Osterholm, Porvoo (FI)

Correspondence Address: Albemarle Netherlands B.V.;Patent and Trademark Department, 451 Florida Street, Baton Rouge, LA, 70801, US

(21) Appl. No.: 11/725,103

(22) Filed: Mar. 16, 2007

(30) Foreign Application Priority Data
Mar. 20, 2006 (EP) 06075690.5

(51) Int. Cl. C07C 5/31; B01J 27/182; B01J 27/185

(52) U.S. Cl. 585/365; 502/213; 502/214

(57) ABSTRACT

A catalyst system for treating a hydrocarbonaceous feed comprising a matrix selected from the group consisting of alumna, silica alumina, titanium alumina and mixtures thereof; a support medium substantially uniformly distributed through said matrix comprising a SAPO-11 molecular sieve; and 0.1 to 1.0 wt % (based on the total weight of the catalyst system) of a catalytically active metal phase supported on said medium and comprising a metal selected from the group consisting of platinum, palladium, ruthenium, rhodium or mixtures thereof. The catalyst system is characterized in that said SAPO-11 molecular sieve has: a) a silica to alumina molar ratio of 0.08 to 0.24; b) a phosphorous to alumina ratio of 0.75 to 0.83; c) a surface area of at least 150 m²/g; d) a crystallite size in the range 250 to 600 angstroms; and, e) a sodium content of less than 2000 ppm weight.

### TECHNICAL BACKGROUND OF THE INVENTION

This invention is concerned with an isomerization catalyst system and with the use of said system in a process for selectively lowering the normal paraffin (n-paraffin) content of a hydrocarbon oil feedstock. In particular, it is concerned with a catalyst system comprising a SAPO-11 silicoaluminophosphate molecular sieve and the use of said system for converting a normal paraffin into a branched paraffin.

### DESCRIPTION OF THE PRIOR ART

Hydrocarbon oil feedstocks boiling in the range from about 177° C. to 700° C. and having a carbon number in the range C₁₅ to C₃₀ find employment inter alia diesel oils and lubricating base oils. For many applications, it is desirable for these components and oils to have low freeze, cloud and/or pour points. For example, the lower the freeze point of a jet fuel, the more suitable it will be for operations under conditions of extreme cold; the fuel will remain liquid and flow freely without external heating even at very low temperatures. In the case of lubricating oils, it is desirable that the pour points be sufficiently low to enable the oil to pour freely—and thereby adequately lubricate—even at low temperature. For example, the pour point of a linear hydrocarbon containing 20 carbon atoms per molecule—having a boiling point of about 340° C. and thereby usually considered as a middle distillate—is about +37° C., rendering it impossible to use as a gas oil for which the specification is −15° C.

Amongst such feedstocks, the market for high paraffinicity oils is continuing to grow due to the high viscosity index (VI), oxidation stability and low volatility (relative to viscosity) of these molecules. However, for applications in which low pour or freeze points are required, it is known that middle-distillate and lube oil range hydrocarbon oils which have high concentrations of normal (n-) paraffins generally have higher freeze points or pour points than oils having lower concentrations of n-paraffins. [Straight chain n-paraffins and only slightly branched chain paraffins are sometimes referred to herein as waxes.] As the n-paraffin component—particularly long chain n-paraffins—imparts undesirable characteristics to oils containing them, they must generally be removed or reduced [by “dewaxing”] in order to produce useful products.

The hydroconversion of n-paraffins to branched paraffins is one of the main routes for producing high octane gasoline blending components, to increase the low temperature performance of diesel and to obtain high viscosity index (VI) lube oils. Although dewaxing by selective cracking of n-paraffins has been extensively used to produce such branched paraffins, cracking can concomitantly degrade useful products to lower value, non-utile lower molecular weight products, such as naptha and gaseous C₁-C₄ products. [The term “naphtha” in used herein to refer to a liquid product having from about C₅ to about C₁₂ carbon atoms in its backbone and which has a boiling range generally below that of diesel, although the upper end of which may overlap that of the initial boiling point of diesel.]

Historically, the need to maximize the isomerisation of n-paraffins while minimizing the undesired (competing) cracking lead to the use of porous silicolauminophosphate (SAPO) as catalysts for hydroisomerisation. SAPOs have a framework of AlO₄, SiO₄ and PO₄ tetrahedra linked by oxygen atoms; the interstitial spaces of the channels formed by the crystalline network enable SAPOs to be used as molecular sieves in a manner similar to crystalline aluminosilicates, such as zeolites.

During hydroisomerisation, the SAPOs' sieve structures can sterically suppress the formation of multi-branched isomers—which are more susceptible to hydrocracking—thereby leading to enhanced isomerisation selectivities. The particular crystalline network of a SAPO molecular sieve determines isomerate shape selectivity: where the pore system of the molecular sieve is sufficiently ‘spacious’, all possible isomers may be formed; conversely, if there are spatial constraints within the sieve, “bulkier” isomers are less prevalent in the product. In general, methyl branching increases with decreasing pore width of the catalyst, whereas ethyl and propyl branched isomers are obtained from wide pore openings and large cavities.

The SAPO pore structure may be selected to enable a given isomerate product to escape the pores quickly enough so that cracking is minimized. For example, U.S. Pat. No. 5,282,958 (Chevron Research and Technology Company) describes a process for the dewaxing of a hydrocarbon feed containing linear paraffins having ≧10 carbon atoms, wherein the feed is contacted under very specific isomerisation conditions with an intermediate pore size molecular sieve—such as SAPO-11, SAPO-31, SAPO-41—having a crystallite size of ≦0.5μ and pores with a diameter between 4.8 and 7.1 angstroms.

The catalyzed hydroisomerisation reaction is carried out in the presence of Lewis acid and base sites within the SAPO molecular sieve, the density of Lewis acid sites commonly being measured by the ion exchange capacity (I.E.C.) of the sieve. The SAPOs are considered to act as bifunctional catalysts, the metallic sites therein facilitating hydrogenation/dehydrogenation and acidic sites catalyzing skeletal isomerisation of n-paraffins (which is considered to proceed via alkylcarbenium ions). The electronegativity of the molecular sieve may be varied by methods known to a person of ordinary skill in the art, such as by modifying the Si/Al ratio within the given range and/or ion exchange.

Nieminen et al. [Applied Catalysis A: General 259 (2004) p. 227-234] describes methods for synthesizing SAPO-11 catalysts of modified acidity by varying the content location and distribution of Si in the molecular sieve. International Patent Application Publication No. WO99/61559 describes the preparation of a molecular sieve having an enhanced silicon: aluminium ratio in which the silicon atoms are distributed such that the number of silicon sites having silicon atoms among all four nearest neighbours is minimized. The SAPO is characterized by having a preferred P/Al molar ratio from 0.9 to about 1.3 and a preferred Si/Al molar ratio of about 0.12 to 0.5.

U.S. Pat. No. 5,817,595 (Tejada et al.) discloses a catalyst system for the hydroisomerisation of a contaminated hydrocarbon feedstock. The system comprises a matrix, a silicoaluminophosphate medium substantially uniformly distributed through the matrix, and a plurality of catalytically active metals from both Group VIB and Group VIII supported on said medium. The catalyst system is further characterized by a surface area of ≧300 m²/g, a crystal size of ≦2 microns and a Si/Al ratio of between 10 and 300.

Ion exchange cations present in the sieve do not form an integral part of the framework, that is, they are not covalently bound into the Si/Al/O network. Thus when taking part in the n-paraffin conversion, it is not necessary for the cations to be removed from the framework and the framework is not weakened. The exchange of cations within the SAPO-11 sieves provides stronger Lewis acid sites. Although trivalent cations may be used in such ion exchanges, the Lewis acid sites produced are generally too strong and therefore it is preferred to use divalent or monovalent cations. Suitable cations include magnesium, calcium, strontium barium, copper, nickel, cobalt, potassium and sodium ions.

There currently exists a need in the art for a catalyst system for the hydroisomerisation that can yield iso-paraffins from waxy feed at a commercially viable conversion rate but which optimizes the balance of Lewis acid and basic sites without the need to necessarily comprise a plurality of catalytically active metal phases.

Petroleum or mineral derived feedstocks which have been isodewaxed using prior art catalyst systems include distillates, raffinates, deasphalted oils and solvent dewaxed oils, said feeds boiling in the range from about 177° C. to 700° C. The hydroisomerisation of feeds which have been pre-treated by hydroprocessing—for example by hydrotreating to remove heteroatom compounds and aromatics—is also known in the art.

Beyond such feeds, U.S. Patent Application No 2003/0057134 (Benazzi et al.) and European Patent Applications No. EP-A-321 303 and EP-A-0 583 836 describe the hydroisomerisation of feeds derived from the Fischer-Tropsch process to obtain middle distillates. In the Fischer-Tropsch process, synthesis gas (CO+H₂) is catalytically transformed into oxygen-containing products and essentially linear gaseous, liquid or solid hydrocarbons, principally constituted by normal paraffins.

The Fischer-Tropsch products are generally free of heteroatomic impurities such as sulphur, nitrogen or metals; they contain low quantities of aromatics, naphthenes and cyclic compounds. However, such products can include significant quantities of oxygen containing and/or unsaturated compounds (particularly olefins). Consequently, although feeds derived from the Fischer-Tropsch process may not require pre-treatment hydrodenitrification (HDN) or hydrodesulfurization (HDS) before hydroisomerisation, they may require catalytic hydrodeoxygenation (HDO).

Recently, attention has focused on the possibility of deriving useful isoparaffins from biological feedstocks, such as a animal or vegetable oils. Given this, there is a need in the art to provide a hydroisomerisation catalyst system that may be utilized effectively with n-paraffinic compounds derived from such sources.

### SUMMARY OF THE INVENTION

In accordance with a first aspect of the present invention there is provided a catalyst system for treating a hydrocarbonaceous feed comprising a matrix selected from the group consisting of alumina, silica alumina, titanium alumina and mixtures thereof; a support medium substantially uniformly distributed through said matrix comprising a SAPO-11 molecular sieve; and 0.1 to 2.0 wt % (based on the total weight of the catalyst system) of a catalytically active metal phase supported on said medium and comprising a metal selected from the group consisting of platinum, palladium, ruthenium, rhodium or mixtures thereof: wherein said catalyst system is characterized in that said SAPO-11 molecular sieve has a) a silica to alumina molar ratio of 0.08 to 0.24; b) a phosphorous to alumina ratio of 0.75 to 0.83; c) a surface area of at least 150 m²/g; d) a crystallite size in the range 250 to 600 angstroms; and, e) a sodium content below 2000 ppm weight. The term hydrocarbonaceous feed is used herein to define any feed which comprises a substantial proportion of linear or slightly branched paraffins.

This catalyst system has been found to be a shape-selective paraffins conversion catalyst which effectively removes normal paraffins from a hydrocarbon oil feedstock by isomerizing them without substantial cracking. The selection of acidity, pore diameter and crystallite size (corresponding to selected pore length) is such as to ensure that there is sufficient acidity to catalyse isomerisation and such that the product can escape the pore system quickly enough so that cracking is minimized. With regard to structure, in accordance with a first preferred embodiment of the invention the silica to alumina ratio of the SAPO-11 molecular sieve is 0.12 to 0.18. Additionally or otherwise, the sodium content of the SAPO-11 molecular sieve is preferably lower than 1000 ppm weight. In accordance with a second preferred embodiment of the invention, said SAPO-11 molecular sieve is further characterized by an average pore volume of at least 0.220 ml/g. Additionally or otherwise it is preferable that the crystallite size of the molecular sieve is in the range from 250 to 500 angstroms.

In accordance with a third preferred embodiment of the invention, the catalytically active metal is platinum. In this case, it is preferable that said catalyst system comprises between 0.1 and 1.0 wt %, and more preferably between 0.3 and 0.7 wt %, of platinum as said catalytically active metal phase.

According to the invention the matrix is selected from the group consisting of alumina, silica alumina, titanium alumina and mixtures thereof, but of which alumina is the most preferred material. This matrix may be porous or non-porous but must be in a form such that it can be combined, dispersed or otherwise intimately admixed with the crystallite molecular sieves. Although it is possible for the matrix itself to be catalytically active, it is preferred that the matrix is not catalytically active in a hydrocracking sense. Irrespective of the matrix activity, it is preferred that the support medium (comprising said SAPO-11) and said matrix (comprising alumina and the like) are present in a ratio by weight of support medium to matrix between 0.1 and 0.8, more preferably between 0.5 and 0.7.

In accordance with a preferred embodiment of this invention, the SAPO-11 molecular sieve is characterized by an ion exchange capacity of at least 400 micromol Si/g (of dried sieve) and more preferably greater than 500 micromol Si/g (of dried sieve). This embodiment is therefore characterized by the close positioning of the active sites within the SAPO-11.

In accordance with a second aspect of the invention, there is provided a process for selectively enhancing the isoparaffin content of a hydrocarbonaceous feed comprising contacting under hydroprocessing conditions said hydrocarbonaceous feed with a catalyst system as defined above. The stocks derived from the process defined in this invention are of high purity, having a high VI, a low pour point and are isoparaffinic, in that they comprise at least 95 wt. % of non-cyclic isoparaffins having a molecular structure in which less than 25% of the total number of carbon atoms are present in the branches, and less than half the branches have two or more carbon atoms.

Although it not essential for the performance of this invention, it is preferred that said hydroprocessing conditions comprise a temperature between 280° C. and 450° C., more preferably between 300° C. and 380° C., a pressure between about 5 and 60 bar, a weight hourly space velocity (WHSV) of from 0.1 hr⁻¹ to about 20 hr⁻¹, and a hydrogen circulation rate of from 150 to 2000 SCF/bbl.

Depending on the nature of the feedstock to be processed, it may be necessary to remove heteroatoms therefrom in order to limit the extent the contamination of the catalyst system. Accordingly, where required the catalyst system may be disposed downstream of a reaction zone in which the hydrocarbonaceous feed is contacted under hydroprocessing conditions with at least one of: an active hydrodeoxygenation (HDO) catalyst, an active hydrodenitrogenation (HDN) catalyst and an active hydrodesulfurization (HDS) catalyst. For spatially efficient commercial processing, these further catalysts may be disposed within a single reactor with said catalyst system.

Definitions

The silica to alumina ratio of the molecular sieves referred to herein may be determined by conventional analysis. This ratio is meant to represent as closely as possible, the ratio in the rigid anionic framework of the silicoaluminophosphate crystal and to exclude aluminum in the matrix material or in cationic or other form within the channels.

The skilled man will be aware that in the preparation of SAPO-11, the silicoaluminophosphate may be contaminated with other SAPOs, and in particular SAPO-41. The term SAPO-11 is here intended to encompass a silicoaluminophosphate of sufficient purity that it exhibits the X-ray diffraction (XRD) pattern characteristic of SAPO-11. (Said X-ray diffraction pattern is demonstrated in Araujo, A. S et al. Materials Research Bulletin Vol. 34, Issue 9, 1 Jul. 1999.)

The length of the crystallite in the direction of the pores (the “c-axis”) is a critical dimension in this invention. For the range of crystallites used, X-ray diffraction (XRD) is the preferred means of measurement of crystallite length. This technique uses line broadening measurements employing the technique described in Klug and Alexander “X-ray Diffraction Procedures” (Wiley, 1954) which is herein incorporated by reference. Thus
D=(K.λ)/(β.cos θ)
Where D=crystallite size (angstroms); K=constant (˜1); λ is wavelength (angstroms) β=corrected half-width in radians; θ=diffraction angle.

The term ion exchange capacity (I.E.C.) is related to the number of active cation sites in the silicoaluminophosphate which exhibit a strong affinity for water molecules and hence appreciably affect the overall capacity of the silicoaluminophosphate to adsorb water vapour. These include all sites which are occupied by any cation, but in any event are capable of becoming associated with sodium or potassium cations when the silicoaluminophosphate is contacted at 25° C. three times for a period of one hour each with a fresh aqueous ion exchange solution containing as the solute 0.2 mole of NaCl or KCl per liter of solution, in proportions such that 100 ml of solution is used for each gram of silicoaluminophosphate. After this contact of the silicoaluminophosphate with the ion-exchange solution, routine chemical gravimetric analysis is performed to determine the relative molar proportions of Al₂O₃, SiO₂ and Na₂O. The data are then substituted in the formula:
I.E.C=k[Na₂O/SiO₂]
wherein ‘k’ is the SiO₂/Al₂O₃ molar ratio of the silicoaluminophosphate immediately prior to contact with the NaCl ion-exchange solution.

### DETAILED DESCRIPTION OF THE INVENTION

The SAPO-11 silicoaluminophosphate molecular sieve for use in the catalyst system of this invention comprises as three-dimensional, microporous crystal framework of corner sharing [SiO₂] tetrahedral, [(AlO₂) tetrahedral and PO₂] tetrahedral units whose empirical formula on an anhydrous basis is:
mR:(SiₓAl_{y}P_{z})O_{z}
wherein “R” represents the at one organic templating agent present in the intracrystalline pore system; “m” represents the moles of “R” present per mole of (mR:(SiₓAl_{y}P_{z})O₂) and has a value from zero to about 0.3; “x”, “y” and “z” represent respectively the mole fractions of silicon, aluminium and phosphorous, said mole fractions being within the relationship defined above.

The unit empirical formula for any SAPO may be given on an “as synthesised” basis relating to SAPO compositions formed as a result of hydrothermal crystallization. Alternatively they may be given after an “as synthesized” SAPO composition has been subjected to a post-treatment process, such as calcination, to remove any volatile components present therein. The reduction in the value of “m” caused by normal post-treatment—thereby precluding treatments which add templates to the SAPO—will depend inter alia on the severity of the post-treatment in terms of its ability to remove the template from the SAPO. Under sufficiently severe post-treatment conditions, e.g., roasting in air at high temperature for long periods (over 1 hr.), the value of “m” may be zero (0) or, in any event, the template, R, is undetectable by normal analytical procedures.

In this invention the SAPO-11 may generally be synthesized by hydrothermal crystallization from a reaction mixture comprising reactive sources of silicon, aluminum and phosphorus, and one or more organic templating agents. Optionally, alkali metal(s) may be present in the reaction mixture. The reaction mixture is placed in a sealed pressure vessel, preferably lined with an inert plastic material, such as polytetrafluoroethylene, and heated, preferably under autogenous pressure at a temperature of at least about 100° C., and preferably between 100° C. and 250° C. until crystals of the silicoaluminophosphate product are obtained, usually for a period of from 2 hours to 2 weeks. While not essential to the synthesis of the SAPO-11, it has been found that stirring or moderate agitation of the reaction mixture and/or seeding the reaction mixture with seed crystals of SAPO-11, or a topologically similar composition, can facilitate the crystallization procedure. The product is recovered by any convenient method such as centrifugation or filtration.

After crystallization the SAPO-11 may be isolated and washed with water and dried in air. As a result of the hydrothermal crystallization, the as-synthesized SAPO contains within its intracrystalline pore system at least one form of the template employed in its formation. Generally, the template is an organic molecular species, but it is possible that at least some of the template is present as a charge-balancing cation. Generally the template cannot move freely through the intracrystalline pore system of the formed SAPO and may be removed by a post-treatment process which (thermally) degrades the template to allow for removal of at least part of it from the SAPO. In some instances, however, the pores of the SAPO may be sufficiently large to permit transport of the template, and, accordingly, complete or partial removal thereof can be accomplished by conventional desorption procedures.

The SAPOs are preferably formed from a reaction mixture having a mole fraction of alkali metal cation which is sufficiently low that it does not interfere with the formation of the SAPO composition. A reaction mixture, expressed in terms of molar oxide ratios, having the following bulk composition is preferred:
aR₂O:(SiₓAl_{y}P_{z})O₂:bH₂O
wherein “R” is a template; “a” has a value great enough to constitute an effective concentration of “R” and is within the range of from greater than zero (0) to about 3; “b” has a value of from zero to 500; “x”, “y” and “z” represent the mole fractions, respectively of silicon, aluminum and phosphors wherein x, y and z each have a value of at least 0.01. The reaction mixture is preferably formed by combining at least a portion of the reactive aluminum and phosphorus sources in the substantial absence of the silicon source and thereafter combining the resulting reaction mixture comprising the aluminum and phosphorus sources with the silicon source. When the SAPOs are synthesized by this method the value of “m” in Formula (1) is generally above about 0.02.

When alkali metal cations are to be included in the SAPO-11, it is preferred to first admix at least a portion of each of the aluminum and phosphorus sources with the alkali metal(s) in the substantial absence of the silicon source. This procedure avoids adding the phosphorus source to a highly basic reaction mixture containing the silicon and aluminum source.

The reaction mixture from which these SAPOs are formed contains one or more organic templating agents described in the art. However, the template preferably at least one alkyl, aryl, aralkyl or arylalkyl and at least one element of Group VA of the Periodic Table, particularly nitrogen, phosphorus, arsenic and/or antimony, more preferably nitrogen or phosphorus and most preferably nitrogen. Nitrogen may be included in the form of mono-, di- and tri-amines, including mixed amines, alone or in combination with a quaternary ammonium compound.

Representative templates, phosphorus, aluminum and silicon sources as well as detailed process conditions are more fully described in U.S. Pat. No. 4,440,871, which is incorporated totally herein by reference.

In accordance with a preferred embodiment of the invention the sodium oxide (Na₂O) content of the silicoaluminophosphate is less than 2000 ppm weight and preferably less than 1000 ppm weight.

When used in the present process, the SAP-11 silicoaluminophosphate molecular sieves are employed in admixture with at least one hydrogenating component selected from the group consisting of platinum, palladium, ruthenium, rhodium or mixtures thereof. The hydrogenating component is included in the SAPO-11 in the range from 0.01 to 1 wt/% based on the weight of the molecular sieve, preferably 0.1 to 5 wt %, more preferably 0.1 to 1% wt % and most preferably 0.3 to 0.7 wt %. Of the primary catalytically active metals listed, platinum and palladium are preferred, of which platinum is the most preferred.

Non-noble metals, such as tungsten, vanadium, molybdenum, nickel, cobalt iron, chromium, and manganese, may optionally be added to the catalyst. However, where these supplementary active metals to be supported on the medium are selected from the group consisting of nickel, cobalt, iron or mixtures thereof the amount of said metal preferably ranges from 0.01 to 6 wt % by weight of the molecular sieve and more preferably from 0.025 to 2.5 wt %. Equally, where the or a further supplementary active metal is selected from the group consisting of tungsten, molybdenum or mixtures thereof, the amount of said metal preferably ranges from 0.01 to 30 wt % by weight of the molecular sieve, more preferably from 10 to 30 wt. %. Within said ranges, combinations of these metals with platinum or palladium, such as cobalt-molybdenum, cobalt-nickel, nickel-tungsten or cobalt-nickel-tungsten, are also useful with many feedstocks.

The techniques of introducing catalytically active metals to a molecular sieve are disclosed in the literature, and preexisting metal incorporation techniques and treatment of the molecular sieve to form an active catalyst are suitable, e.g., ion exchange, impregnation or by occlusion during sieve preparation. See, for example, U.S. Pat. Nos. 3,236,761; 3,226,339; 3,236,762; 3,620,960; 3,373,109; 4,202,996; and 4,440,871 which patents are incorporated totally herein by reference.

The hydrogenation metal included in the catalyst system of this invention can mean one or more of the metals in its elemental state or in a form such as the sulfide or oxide and mixtures thereof. As is well-known, references to the active metal is intended to encompass the existence of such metal in the elemental state or as a compound thereof but regardless of the state in which the metallic component actually exists, the concentrations are computed as if they existed in the elemental state.

The physical form of the silicoaluminophosphate depends on the type of catalytic reactor being employed but typically is in the form of a granule or powder as this facilitates its compaction into a usable form (e.g. larger agglomerates) with the matrix material.

Compositing the crystallites with an inorganic oxide matrix can be achieved by any suitable known method wherein the crystallites are intimately admixed with the oxide while the latter is in a hydrous state (for example, as a hydrous salt, hydrogel, wet gelatinous precipitate) or in a dried state, or combinations thereof. A conventional method is to prepare a hydrous mono or plural oxide gel or cogel using an aqueous solution of a salt or a mixture of salts (for example aluminium and sodium silicate). Ammonium hydroxide carbonate or a similar base is added to the solution in an amount sufficient to precipitate the oxides in hydrous form. Then the precipitate is washed to remove most of the any water soluble salts and it is thoroughly admixed with the crystallites. Water or lubricating agent can be added in an amount sufficient to facilitate shaping of the mix. The combination can then be partially dried as desired, tableted, pelleted, extruded or formed by other means and then calcined, for example, at a temperature above 316° C. and more usually at a temperature above 427° C. Processes which produce larger pore size supports are preferred to those producing smaller pore size supports when cogelling.

According to the invention the matrix is selected from the group consisting of alumina, silica alumina, titanium alumina and mixtures thereof. This matrix may be porous or non-porous but must be in a form such that it can be combined, dispersed or otherwise intimately admixed with the crystallite molecular sieves. Although it is possible for the matrix itself to be catalytically active—for example to facilitate cracking of the longer chain n-paraffins—it is preferred that the matrix is not catalytically active in a hydrocracking sense.

The derived catalyst system may be employed either as a fluidized catalyst, or in a fixed or moving bed, and in one or more reaction stages.

The feedstocks which can be treated in accordance with the present invention include oils which generally have a high pour points which it desired to reduce to relatively low pour points. The isomerisation catalyst system of this invention may thus be used to reduce the n-paraffin content of a variety of high boiling stocks [such as whole crude petroleum, reduced crudes, vacuum tower residua, cycle oils and synthetic crudes]; middle distillate feedstocks [including gas oils, kerosenes, and jet fuels, lubricating oil stocks, heating oils and other distillate fractions whose pour point and viscosity need to be maintained within certain specification limits]; synthetic oils [such as those produced by Fischer-Tropsch synthesis, high pour point polyalphaolefins, foot oils, synthetic waxes such as normal alphaolefin waxes, slack waxes, deoiled waxes and microcrystalline waxes]; and, lighter distillates containing normal paraffins such as straight run gasoline or gasoline range fractions from hydrocracking. Hydroprocessed stocks are a convenient source of lubricating oil stocks and also of other distillate fractions since they normally contain significant amounts of waxy n-paraffins. The feedstock can generally be a C10+ feedstock boiling at about 175°—since lighter oils will usually be free of significant quantities of waxy components—but is more preferably a C15+ feedstock boiling above 230° C. Although the feedstock may comprise olefins, naphthenes, aromatics and heterocyclic compounds, it is preferred that the feedstock comprises a substantial proportion of high molecular weight n-paraffins and slightly branched paraffins which contribute to the waxy nature of the feedstock.

In accordance with a preferred embodiment of the invention, the feed comprises a substantial proportion of n-paraffins in the range C₁₅ to C₁₀₀. More preferably, the feedstock comprises from 70 to 100 wt % C₁₅ to C₄₀ linear paraffins and most preferably 85 to 95 wt % C₁₅ to C₄₀ linear paraffins.

It is well known that nitrogen and sulphur contaminants in non-biological feedstocks tend to rapidly deactivate process catalysts and, furthermore, are undesirable fractions in the final product. In accordance with the process of this invention, non-biological feedstocks to be treated preferably have a sulphur content less than 10,000 ppmw and a nitrogen content less than 200 ppmw. More preferably, non-biological feedstocks should have an organic nitrogen content of less than 100 ppmw. Equally, feeds derived from synthetic or biological feedstocks—such as those derived from treated animal or vegetable fats—may comprise a contaminating level of oxygen containing and/or unsaturated species. Preferably the oxygen and/or unsaturated olefin content of the feed is less than 200 ppmw.

In order to reduce the level of sulphur and nitrogen and of oxygen or unsaturated contaminants in the feed it may be necessary to pre-treat the feed before it is subjected to hydroisomerisation. The feed may therefore undergo hydrodenitrification (HDN), hydrodesulfurization (HDS) and/or hydrodeoxygenation (HDO). The person of ordinary skill in the art would be aware of a number of treatments that could be applied to achieve these effects. Preferably, however, where the feed is preheated, this is effected using catalytic hydroprocessing; this makes it possible for a first catalytic hydroprocess to be positioned downstream of the hydroisomerisation process; such a downstream position may optionally be within the same reactor through which the feed is (directionally) passed.

The hydroisomerisation conditions to be used in accordance with the present invention will of course vary depending upon the exact catalyst and feedstock to be used and the final product which is desired. However said conditions include a temperature in the range from 200° C. to 400° C., a pressure in the range 1 to 200 bar. More preferably the pressure is from about 5 to 80 bar and most preferably 30 to 70 bar. The weight hourly space velocity (WHSV) is generally in the range between 0.1 and 20 hr⁻¹ during contacting with the catalyst but is more preferably in the range from 0.5 to 5 hr⁻¹.

In that preferred embodiment wherein said contacting occurs in the presence of hydrogen, the hydrogen to hydrocarbon ratio generally falls in the range from 1 to 50 moles H₂ per mole hydrocarbon and more preferably from 10 to 30 moles H₂ per mole hydrocarbon.

The process of the present invention may also be used in combination with conventional dewaxing processes to achieve an oil having desired properties. Such processes may be employed prior to or immediately after the isomerisation process of the invention. Further, the pour point of the hydroisomerate produced by the process of the present invention may also be reduced by adding pour point depressant compositions thereto.

For higher boiling waxy feeds, after said feed has been hydroisomerized, the hydroisomerate may be sent to a fractionater to remove the 650-750° F.—boiling fraction and the remaining 650-750° F.+hydroisomerate dewaxed to reduce its pour point and form a dewaxate comprising the desired lube oil base stock. If desired however, the entire hydroisomerate may be dewaxed. If catalytic dewaxing is used, that portion of the 650-750° F.+material converted to lower boiling products is removed or separated from the 650-750° F.+lube oil base stock by fractionation, and the 650-750° F.+dewaxate fractionated separated into two or more fractions of different viscosity, which are the base stocks of the invention. Similarly, if the 650-750° F. material is not removed from the hydroisomerate prior to dewaxing, it is separated and recovered during fractionation of the dewaxate into the base stocks.

The product of the present invention may be further treated as by hydrofinishing. The hydrofinishing can be conventionally carried out in the presence of a metallic hydrogenation catalyst, for example, platinum on alumna. The hydrofinishing can be carried out at a temperature of from about 190° C. to about 340° C., and a pressure of from about 400 psig to about 3000 psig. Hydrofinishing in this manner is described in, for example, U.S. Pat. No. 3,852,207 which is incorporated herein by reference.

The following examples further illustrate the preparation and use of the catalyst system according to the invention.

### EXAMPLES

Four different SAPO-11 materials were prepared having the properties shown in Table 1. Of these, SAPO-11-A and SAPO-11-D possess the characterizing features required for employment in the catalyst system of this invention. Those features of SAPO-11-B and SAO-11-C which do not meet these characterizing requirements are highlighted in this table.

TABLE 1

| | SAPO- | SAPO- | SAPO- | SAPO- |
|---|---|---|---|---|
| Description | 11-A | 11-B | 11-C | 11-D |
| Si mole | 0.13 | 0.17 | 0.08 | 0.10 |
| P mole | 0.68 | 0.60 | 0.71 | 0.69 |
| Al mole | 0.88 | 0.86 | 0.86 | 0.87 |
| (Si + P) mole | 0.81 | 0.78 | 0.77 | 0.79 |
| Si/Al ratio | 0.14 | 0.20 | 0.09 | 0.12 |
| P/Al ratio | 0.78 | 0.71 | 0.83 | 0.80 |
| N₂-SA-BET (m²/g) | 235 | 205 | 202 | 244 |
| N₂-PV-Ads (ml/g) | 0.229 | 0.154 | 0.154 | 0.212 |
| MiPV (3-5) (ml/g) | 0.069 | 0.048 | 0.086 | 0.056 |
| Micro SA (m²/g) | 173 | 136 | 171 | 174 |
| Crystallite Size | 392 | 400 | 630 | 360 |
| (Angstroms) | | | | |
| Na₂O content (ppm) | 800 | 1700 | 350 | 970 |

Four hydroisomerisation catalyst systems (A, B, C and D) were then prepared using these SAPO-11 samples. Firstly, extrusion mixtures were prepared by combining 30 wt. % boehmite alumina and 70 wt. % of the relevant SAPO-11 material, to which were then added a small amount of nitric acid and cellulose to act as extrusion agents. The mixtures were then extruded using a Killion extruder in a 1.5E cylindrical shape, the extrudates dried at 120° C. overnight and subsequently calcined for 1 hour at 550° C.

The products so-obtained were then loaded with 0.5 wt. % Pt using a 3% tetra-amine platinum (II) nitrate solution and calcined in air for two hours at 450° C. to yield the four catalyst systems defied in Table 2.

TABLE 2

| | Catalyst A | Catalyst B | Catalyst C | Catalyst D |
|---|---|---|---|---|
| SAPO-11 | SAPO-11-A | SAPO-11-B | SAPO-11-C | SAPO-11-D |
| Pt (wt %) | 0.495 | 0.490 | 0.509 | 0.502 |
| N₂-SA-BET | 200 | 203 | 231 | 210 |
| (m²/g) | | | | |
| N₂-PV Ads | 0.277 | 0.299 | 0.170 | 0.258 |
| (ml/g) | | | | |

### Example 1

Catalyst systems A and B were tested in fixed bed reactor for the hydroisomerisation of a feed consisting of 100% linear paraffins having carbon numbers in the range C15 to C18. The test conditions employed were: temperature 340° C.; pressure 60 Bar; weight hourly space velocity (WHSV) 3 h⁻¹; and, a hydrogen to feed ratio of 600 l/l.

The hydroisomerates obtained by contacting the feed with the respective catalyst systems had the properties shown in Table 3.

TABLE 3

| | Catalyst A | Catalyst B |
|---|---|---|
| Cloud Point (° C.) | −24 | 21 |

The cloud point of the hydroisomerate obtained by contacting the feed with catalyst system A is significantly lower than those cloud points for the hydroisomerates obtained by contacting the same feed with the comparative catalyst system B.

### Example 2

Catalyst systems C and D were tested in fixed bed reactor for the hydroisomerisation of a feed consisting of 100% linear paraffins (derived from animal fat) having carbon numbers in the range C15 to C18. The test conditions employed were: temperature 318° C.; pressure 40 Bar; weight hourly space velocity (WHSV) 1.5 hr⁻¹; and, a hydrogen to feed ratio of 300 l/l.

The hydroisomerates obtained by contacting the feed with the respective catalyst systems had the properties shown in Table 4.

TABLE 4

| | Catalyst C | Catalyst D |
|---|---|---|
| Cloud Point (° C.) | −4 | −20 |

The cloud point of the hydroisomerate obtained by contacting the feed with catalyst system D is significantly lower than those cloud points for the hydroisomerates obtained by contacting the same feed with the comparative catalyst system C.

It is understood that various other embodiments and modifications in the practice of the invention will be apparent to, and can be readily made by, those skilled in the art without departing from the scope and spirit of the invention described above.

1. A catalyst system for treating a hydrocarbonaceous feed comprising a matrix selected from the group consisting of alumina, silica alumina, titanium alumina and mixtures thereof; a support medium substantially uniformly distributed through said matrix comprising a SAPO-11 molecular sieve; and 0.1 to 1.0 wt % (based on the total weight of the catalyst system) of a catalytically active metal phase supported on said medium and comprising a metal selected from the group consisting of platinum, palladium, ruthenium, rhodium or mixtures thereof:
wherein said catalyst system is characterized in that said SAPO-11 molecular sieve has;
a) a silica to alumina molar ratio of 0.08 to 0.24;
b) a phosphorous to alumina ratio of 0.75 to 0.83;
c) a surface area of at least 150 m²/g;
d) a crystallite size in the range 250 to 600 angstroms; and
e) a sodium content of less than 2000 ppm weight.

2. The catalyst system according to claim 1, wherein said matrix is not catalytically active.

3. The catalyst system according to claim 1 or claim 2, wherein said matrix comprises alumina.

4. The catalyst system according to any one of claims 1 to 3, wherein said SAPO-11 molecular sieve is further characterized by an ion exchange capacity (I.E.C.) of at least 600 micromol. Si/g.

5. The catalyst system according to any one of claims 1 to 4, wherein said SAPO-11 molecular sieve is further characterized by an average pore volume of at least 0.220 ml/g.

6. The catalyst system according to any one of claims 1 to 5, wherein said catalytically active metal is platinum.

7. The catalyst system according to claim 6, comprising 0.3 to 2.0 wt % of platinum as said catalytically active metal phase.

8. The catalyst system according to any one of claims 1 to 7, further comprising 0.01 to 6.0 wt % of a supplementary active metal phase supported on said matrix and comprising a metal selected from the group consisting of nickel, cobalt, iron and mixtures thereof.

9. The catalyst system according to any one of claims 1 to 8, further comprising 10 to 30 wt % of a supplementary active metal phase supported on said matrix and comprising a metal selected from the group consisting of tungsten, molybdenum and mixtures thereof.

10. The catalyst system according to any one of claims 1 to 9, wherein said support medium and said matrix are present in a ratio by weight of support medium to matrix between 0.1 and 1.0.

11. A process for selectively enhancing the isoparaffin content of a hydrocarbonaceous feed comprising contacting under hydroprocessing conditions said hydrocarbonaceous feed with a catalyst system as defined in any one of claims 1 to 10.

12. The process according to claim 11, wherein said hydroprocessing conditions comprise a temperature between 280° C. and 450° C., a pressure between about 5 and 60 bar, a liquid hourly space velocity of from 0.1 hr⁻¹ to about 20 hr⁻¹, and a hydrogen circulation rate of from 150 to 2000 SCF/bbl.

13. The process according to claim 11 or claim 12, wherein said hydrocarbonaceous feed substantially comprises C15 to C40 linear paraffins.

14. A process according to claim 12 or claim 13, wherein said catalyst system is disposed downstream of a reaction zone in which the hydrocarbonaceous feed is contacted under hydroprocessing conditions with at least one of: an active hydrodeoxygenation (HDO) catalyst, an active hydrodenitrogenation (HDN) catalyst and an active hydrodesulfurization (HDS) catalyst.

15. The process according to claim 14, wherein said at least one her catalysts are disposed in a single reactor with said catalyst system.

16. The process according to claim 14 or claim 15, wherein said hydrocarbonaceous feed is of biological origin, preferably comprising animal or vegetable oils or mixtures thereof.

17. The process according to claim 16, wherein said hydrocarbonaceous feed comprises rapeseed oil, palm oil, soybean oil, tallow, animal fat or mixtures thereof.
